(19) United States
(12) Patent Application Publication
(10) Pub. No.: US 2016/0022695 A1
(43) Pub. Date: Jan. 28, 2016

(54) Bimatoprost Ocular Silicone Inserts and Methods of Use Thereof

(71) Applicant: FORSIGHT VISION5, INC., Menlo Park, CA (US)

(72) Inventors: Cary Reich, Menlo Park, CA (US); Carlos Schuler, Cupertino, CA (US); Qiang Ye, San Diego, CA (US); Logan Stark, Sunnyvale, CA (US); Rachna Jain, Fremont, CA (US)

(21) Appl. No.: 14/775,989

(22) PCT Filed: Mar. 27, 2014
(86) PCT No.: PCT/US2014/031955

§ 371 Date: Sep. 14, 2015

Related U.S. Application Data
(60) Provisional application No. 61/805,895, filed on Mar. 27, 2013.

(51) Int. Cl. A61K 31/5575; A61K 9/00; A61K 47/34

(52) U.S. Cl. CPC A61K 31/5575; A61K 47/34; A61K 9/0051

(57) ABSTRACT

The present invention is directed to compositions of bimatoprost, processes of preparing these compositions, devices comprising these compositions, and methods of lowering intraocular pressure.

### RELATED APPLICATIONS

This application claims priority to, and the benefit of, U.S. provisional application No. 61/805,895, filed Mar. 27, 2013, the entire content of which is incorporated herein by reference in its entirety.

### FIELD OF INVENTION

Embodiments disclosed herein are generally directed to compositions comprising a polymer matrix and stable amorphous bimatoprost, wherein the bimatoprost is dispersed in the polymer matrix

### BACKGROUND OF THE INVENTION

Intraocular pressure (IOP) is the fluid pressure inside the eye. IOP is an important aspect in the evaluation of patients at risk from glaucoma, which is a progressive optic neuropathy that can cause blindness. Bimatoprost (7-[3,5-dihydroxy-2-(3-hydroxy-5-phenyl-pent-1-enyl)-cyclopentyl]-N-ethyl-hept-5-enamide) is currently marketed as an ophthalmic solution, by Allergan (LUMIGAN®) and is useful for the treatment of open-angle glaucoma and ocular hypertension. When an ophthalmic solution containing bimatoprost is administered to the eye, the patient may experience side effects, e.g., blurred vision, eyelid redness, permanent darkening of the eyelashes, eye discomfort, permanently darkening the iris (to brown), temporary burning sensation during use, growth and/or thickening of the eyelashes, unexpected growth of hair (if applied inappropriately on the skin), or darkening of the eyelid or of the area beneath the eye. Further, application of such a solution does not provide sustained release of bimatoprost into the eye. Accordingly, new compositions of bimatoprost are needed. The present invention addresses these needs.

### SUMMARY OF THE INVENTION

In one aspect, the present invention features a composition comprising a polymer matrix and stable amorphous bimatoprost, wherein the bimatoprost is dispersed in the polymer matrix.

In some embodiments, the polymer matrix comprises a thermoplastic polymer that is processed after the bimatoprost and the thermoplastic polymer are mixed. The processing is performed by heating at an elevated temperature. The processing temperature can be above the melting point of bimatoprost. For example, the processing temperature is equal to or above about 65° C., or equal to or above about 100° C., or equal to or above about 140° C. to about 160° C., or about 152° C.

In some embodiments, the polymer matrix comprises a thermosetting polymer that is cured after the bimatoprost and the uncured thermosetting polymer are mixed. An example of the thermosetting polymer is silicone, such as MED-4810, MED-4820, MED-4830, MED-4840, MED-4842, MED1-4855, MED-4860, MED-4870, or MED-4880. The curing is performed by heating at an elevated temperature. The curing temperature can be above the melting point of bimatoprost. For example, the curing or processing temperature is equal to or above about 65° C., or equal to or above about 100° C., or equal to or above about 140° C. to about 160° C., or about 152° C.

In some embodiments, the composition of this invention is configured as a medical device, e.g., a device intended to be place on or in the eye. In some instances, the device has a ring shape. The diameter of the ring can be about 10 mm to about 40 mm or about 20 mm to about 30 mm and the cross-sectional thickness is about 0.1 mm to about 5 mm or about 0.5 mm to about 1.5 mm.

In some embodiments, bimatoprost is about 1% to about 30% by weight, about 2% to about 30% by weight, about 2% to about 25% by weight, about 2% to about 22% by weight of the composition. In some instances, the bimatoprost is about 2%, about 3%, about 4%, about 5%, about 6%, about 7%, about 8%, about 10%, about 11%, about 12%, about 13%, about 14%, about 15%, about 16%, about 17%, about 18%, about 19%, about 20%, about 21%, or about 22% by weight of the composition. In some embodiments, bimatoprost is about 2% by weight.

In one embodiment, the ocular insert comprising a polymer matrix and stable amorphous bimatoprost of the current disclosure includes at least one secondary therapeutic agent. For example, the secondary agent may be Loteprednol (loteprednol etabonate) and/or Timolol (Timolol maleate).

In some embodiments, the ocular insert comprising a polymer matrix and stable amorphous bimatoprost of the current disclosure includes additives and/or excipients. For example, such additives or excipients can be phospholipid (e.g., 1,2-dimyristoyl-sn-glycero-3-phosphocholine (DMPC)), stearyl alcohol, and/or carbopol.

In another aspect, the present invention features a method of preparing a composition comprising: dissolving bimatoprost in an organic solvent to form a solution, mixing the solution with an uncured polymer, removing the organic solvent, and curing the polymer at a temperature above the melting point of bimatoprost.

In some embodiments of the above method, the curing temperature is equal to or above about 65° C., equal to or above about 100° C., equal to or above about 140° C. to about 160° C., or about 152° C. In some embodiments, the polymer is silicone. In further embodiments, the organic solvent is selected from dichloromethane, chloroform, acetone, acetonitrile, methanol, ethanol, isopropanol, ethyl acetate, diethyl ether, and a mixture thereof. In some instances, the organic solvent is a non-polar solvent, such as dichloromethane, chloroform, ethyl acetate, diethyl ether, or a mixture thereof. For example, the solvent is dichloromethane. In some embodiments, the curing step lasts for about 2 minutes to about 10 minutes, e.g., about 5 minutes. In certain embodiments, before the curing step, the resulting mixture from the removing step is shaped.

The method may further include one or both (1) washing the composition with water or an organic solvent and (2) sterilizing the composition, e.g., by radiation with electron beam.

In another aspect, the invention features a device comprising any composition described above.

In another aspect, the invention features a composition prepared according to the method and various embodiments described above. For example, in one embodiment, the invention features a composition prepared by a process comprising heating bimatoprost at about 65° C. to about 80° C. to form a melt, mixing the melt with an uncured polymer at a ratio of about 2% to about 20% in one or more cycles for about 5 minutes to 1 hour each, and curing the polymer at about 60° C. to about 160° C. for about 3 minutes to about 7 minutes. In one embodiment, the process comprises heating bimatoprost at about 70° C. to form a melt, mixing the melt with an uncured polymer at a ratio of about 20% in one or more cycles for about 30 minutes each, and curing the polymer at about 150° C. for about 5 minutes.

In another embodiment, for example, the invention features a composition prepared by a process comprising mixing bimatoprost powder with an uncured polymer at a ratio of about 2% to about 30% in one or more cycles for about 1 minute to about 1 hour each; while mixing, heating the mixture at about 65° C. to about 80° C. to melt said bimatoprost into the polymer, in one or more cycles for about 5 minutes to about 1 hour each; and curing the polymer at a temperature at about 60° C. to about 160° C., for about 3 minutes to about 7 minutes. In one embodiment, the process comprises mixing bimatoprost powder with an uncured polymer at a ratio of about 20% in one or more cycles for about 30 minutes each; while mixing, heating the mixture at about 70° C. to melt the bimatoprost into the polymer, in one or more cycles for about 30 minutes each; and curing the polymer at a temperature at about 150° C., for about 5 minutes.

In another aspect, the invention features a method of using the composition described herein to treat diseases, e.g., to lower intraocular pressure.

Other features and advantages of the invention will be apparent from the following detailed description and claims.

### BRIEF DESCRIPTION OF THE FIGURES

FIG. 1 shows an embodiment of the ocular device of the present invention and its cross-sectional view.

FIG. 2 shows a scheme of manufacturing the ocular device of the present invention.

FIG. 3 demonstrates administration of the ocular device of the present invention to a patient.

FIG. 4 is an X-ray powder diffraction pattern of commercially available crystalline bimatoprost.

FIG. 5 is an X-ray powder diffraction pattern of cured Part A and Part B of MED-4810 silicone.

FIG. 6 is an X-ray powder diffraction pattern of 7% of bimatoprost in Part A and Part B of MED-4810 silicone before curing.

FIG. 7 is an X-ray powder diffraction pattern of 7% of bimatoprost in Part A and Part B of MED-4810 silicone cured at 305° F. (about 152° C.) for 5 minutes.

FIG. 8 shows the differential scanning calorimetry profiles of 7% bimatoprost in Part A and Part B of MED-4810 at 10° C./min under nitrogen.

FIG. 9 is an X-ray powder diffraction pattern of 20% of bimatoprost in Part A and Part B of MED-4830 silicone before curing.

FIG. 10 is an X-ray powder diffraction pattern of 20% of bimatoprost in Part A and Part B of MED-4830 silicone cured at room temperature.

FIG. 11 is an X-ray powder diffraction pattern of 20% of bimatoprost in Part A and Part B of MED-4830 silicone cured at 305° F. for 5 minutes.

FIG. 12 is an X-ray powder diffraction pattern of a cured MED-4830 without bimatoprost.

FIG. 13 is an X-ray powder diffraction pattern of 20% of bimatoprost in cured MED-4830 silicone, after being washed and irradiated by electron beam.

FIG. 14 is an X-ray powder diffraction pattern of 20% of bimatoprost in cured MED-4830 silicone, after being washed and irradiated by electron beam, and then eluted at 37° C. in saline for 179 days.

FIG. 15 is an X-ray powder diffraction pattern of 7% of bimatoprost in Part A and Part B of MED-4810 silicone cured at 305° F. for 5 minutes and eluted for 148 days.

FIG. 16 is an X-ray powder diffraction pattern of 7% of bimatoprost in Part A and Part B of MED-4810 silicone cured at 305° F. for 5 minutes and stored for 9 months at 40° C. and 75% relative humidity.

FIG. 17 is a flowchart of the process scheme in which bimatoprost is melted prior to mixing with a polymer in the manufacture of the ocular device of the present invention.

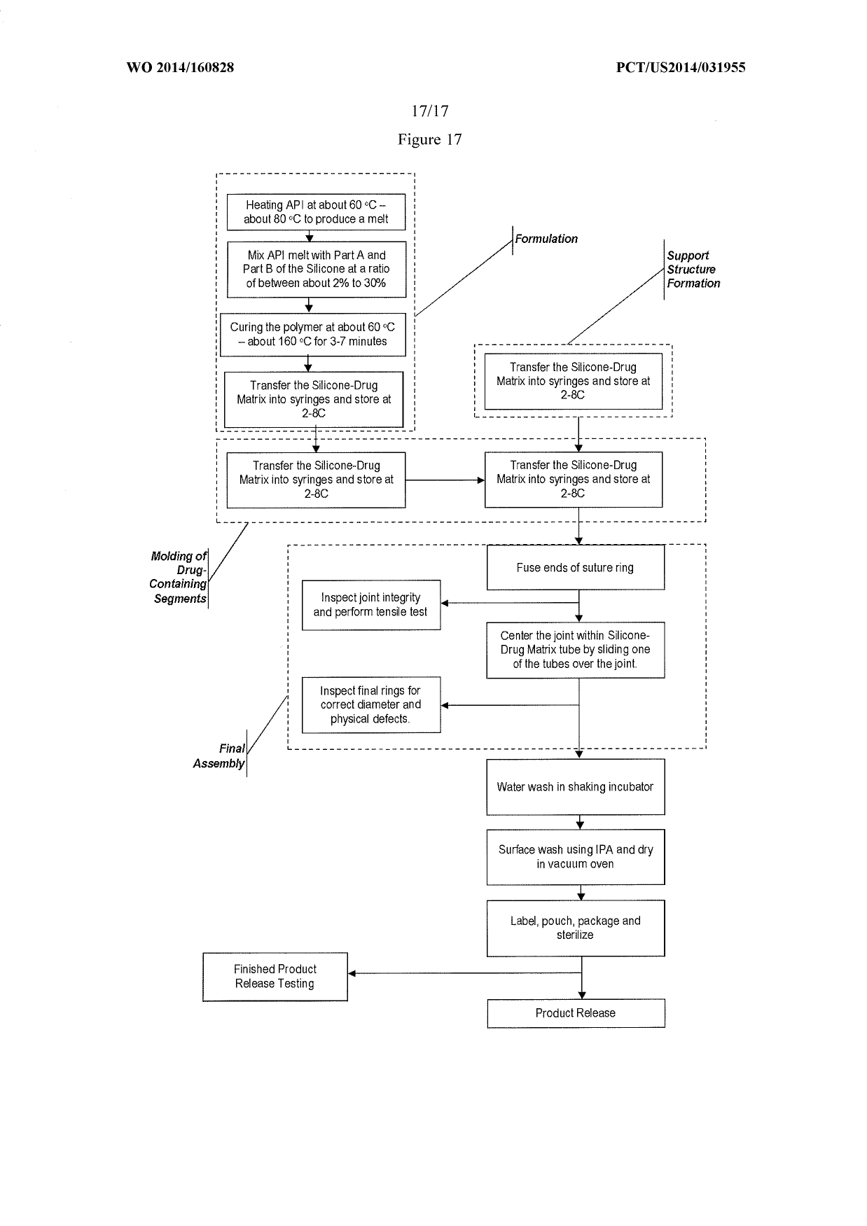

### DETAILED DESCRIPTION OF THE INVENTION

The present invention is directed to compositions of bimatoprost, process of preparing these compositions, medical devices comprising these compositions, and methods of lowering intraocular pressure using the composition described herein.

### Compositions

The present compositions provide for sustained release of bimatoprost to the eye. The sustained release of bimatoprost may be for a long period of time. The therapeutic efficacy of bimatoprost may be improved by increasing its contact time with the corneal surface. The conventional ocular dosage forms for the delivery of bimatoprost are ophthalmic solution or ointments. Although the eye drop dosage form (solution) may be easy to administer, this administration has disadvantages since most of the instilled volume is eliminated from the pre-corneal area, resulting in poor bioavailability. This may occur due to conjunctival absorption, rapid solution drainage by gravity, induced lachrymation, blinking reflex, low corneal permeability, and normal tear turnover. Frequent instillations of eye solution may be necessary to maintain a continuous sustained therapeutic level. By contrast, the compositions of this invention may not require frequent administration to maintain a continuous sustained therapeutic level. Further, the compositions of the present invention may not produce side effects associated with solution administration such as blurred vision, eyelid redness, permanent darkening of eyelashes, eye discomfort, permanent darkening of iris (to brown), temporary burning sensation during use, growth and/or thickening of the eyelashes, unexpected growth of hair (if applied inappropriately on the skin), darkening of the eyelid or of the area beneath the eye.

The present invention provides a stabilized amorphous form of bimatoprost. Surprisingly, a substantial amount (50% or more, 60% or more, 70% or more, 80% or more, 90% or more, or 95% or more) of bimatoprost retains amorphous form in the polymer matrix after 1 day, 30 days, 60 days, 90 days, 120 days, 150 days, 180 days, or even longer. The present invention also provides a stable bimatoprost composition. It has been found that the bimatoprost in the compositions of this invention remains in the stable amorphous form for a long period time, even while exposed to humidity and stored or used at 37° C.

The compositions of this invention comprise a polymer matrix and stable amorphous bimatoprost, wherein the bimatoprost is dispersed in the polymer matrix. In some embodiments, the polymer matrix comprises a thermoplastic polymer that is processed by mixing the bimatoprost and the thermoplastic polymer at elevated temperature. Examples of thermoplastic polymer include, but are not limited to, acrylonitrile butadiene styrene (ABS), acrylic (PMMA), celluloid, cellulose acetate, cycloolefin copolymer (COC), ethylene-vinyl acetate (EVA), ethylene vinyl alcohol (EVOH), fluoroplastics (PTFE, alongside with FEP, PFA, CTFE, ECTFE, ETFE), ionomers, Kydex, liquid crystal polymer (LCP), polyacetal (POM or Acetal), polyacrylates (Acrylic), polyacrylonitrile (PAN or Acrylonitrile), polyamide (PA or Nylon), polyamide-imide (PAI), polyaryletherketone (PAEK or Ketone), polybutadiene (PBD), polybutylene (PB), polybutylene terephthalate (PBT), polycaprolactone (PCL), polychlorotrifluoroethylene (PCTFE), polyethylene terephthalate (PET), polycyclohexylene dimethylene terephthalate (PCT), polycarbonate (PC), polyhydroxyalkanoates (PHAs), polyketone (PK), polyester, polyethylene (PE), polyetheretherketone (PEEK), polyetherketoneketone (PEKK), polyetherimide (PEI), polyethersulfone (PES), polyethylenechlorinates (PEC), polyimide (PI), polylactic acid (PLA), polymethylpentene (PMP), polyphenylene oxide (PPO), polyphenylene sulfide (PPS), polyphthalamide (PPA), polypropylene (PP), polystyrene (PS), polysulfone (PSU), polytrimethylene terephthalate (PTT), polyurethane (PU), polyvinyl acetate (PVA), polyvinyl chloride (PVC), polyvinylidene chloride (PVDC), and styrene-acrylonitrile (SAN).

In other embodiments, the polymer matrix comprises a thermosetting polymer that is cured after the bimatoprost and the uncured thermosetting polymer are mixed. Examples of suitable thermosetting polymers include, but are not limited to, silicones (e.g. MED-4800 series such as MED-4810, MED-4820, MED-4830, MED-4840, MED-4842, MED1-4855, MED-4860, MED-4870, or MED-4880), polyesters (e.g. PET), polyurethanes, vulcanized rubbers, urea-formaldehyde, melamine, epoxy, polyimides, cyanate esters (polycyanurates), vinylesters, bakelite (a phenol-formaldehyde), and duroplast (similar to bakelite).

The curing or processing is performed by heating at an elevated temperature. The curing or processing temperature can be above the melting point of bimatoprost. For example, the curing or processing temperature is equal to or above about 65° C., equal to or above about 80° C., equal to or above about 100° C., equal to or above about 140° C. to about 160° C., or about 152° C.

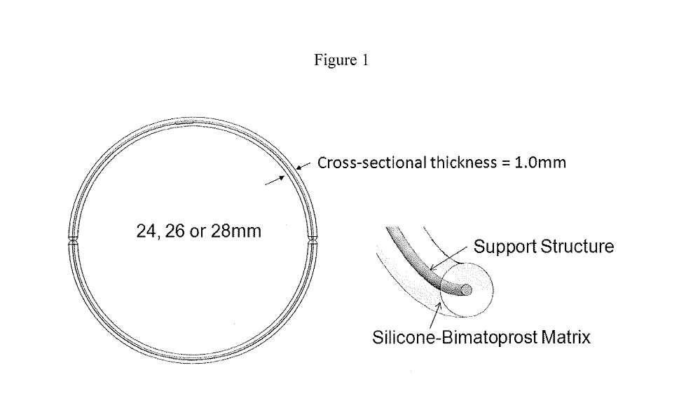

The compositions of this invention may be configured as a device, e.g., a medical device. The medical device can be an ocular insert intended to be placed on or in the eye. In some embodiments, the ocular insert has a ring shape. The diameter of the ring can be about 10 mm to about 40 mm or about 20 mm to about 30 mm (e.g., about 20 mm, about 21 mm, about 22 mm, about 23 mm, about 24 mm, about 25 mm, about 26 mm, about 27 mm, about 28 mm, about 29 mm, or about 30 mm) and the cross-sectional thickness can be 0.1 mm to about 5 mm or about 0.5 mm to about 1.5 mm (e.g., about 0.5 mm, about 0.6 mm, about 0.7 mm, about 0.8 mm, about 0.9 mm, about 1 mm, about 1.1 mm, about 1.2 mm, about 1.3 mm, about 1.4 mm, or about 1.5 mm). FIG. 1 demonstrates an embodiment of the ring-shaped insert.

In the compositions of this invention, bimatoprost may be about 0.1% to about 40% by weight of the composition, about 1% to about 30% by weight of the composition, about 2% to about 30% by weight of the composition, about 2% to about 25% of the composition, or about 2% to about 22% by weight of the composition. In certain instances, the bimatoprost is about 2%, about 3%, about 4%, about 5%, about 6%, about 7%, about 8%, about 10%, about 11%, about 12%, about 13%, about 14%, about 15%, about 16%, about 17%, about 18%, about 19%, about 20%, about 21%, or about 22% by weight of the composition.

The compositions of this invention may include at least one second therapeutic agent. Examples of such an agent include, but are not limited to, a muscarinic agent, a beta blocker, an alpha agonist, a carbonic anhydrase inhibitor, another prostaglandin analog, an anti-inflammatory agent, an anti-infective agent, a dry eye medication, or any combination thereof. See, e.g., U.S. Patent Application Publication 2009/0104243. In one embodiment, the secondary therapeutic agent used in an ocular insert comprising a polymer matrix and stable amorphous bimatoprost is Loteprednol (loteprednol etabonate) and/or Timolol (Timolol maleate).

In some embodiments, at least one second therapeutic agent included in the compositions of the current invention may be an anti-glaucoma agent. Suitable anti-glaucoma therapeutic agents include: sympathomimetics such as Apraclonidine, Brimonidine, Clonidine, Dipivefrine, and Epinephrine; parasympathomimetics such as Aceclidine, Acetylcholine, Carbachol, Demecarium, Echothiophate, Fluostigmine, Neostigmine, Paraoxon, Physostigmine, and Pilocarpine; carbonic anhydrase inhibitors such as Acetazolamide, Brinzolamide, Diclofenamide, Dorzolamide, and Methazolamide, beta blocking agents such as Befunolol, Betaxolol, Carteolol, Levobunolol, Metipranolol, and Timolol; additional prostaglandin analogues such as Latanoprost, Travoprost, and Unoprostone; and other agents such as Dapiprazole, and Guanethidine.

In additional embodiments, the secondary agent for delivery from the delivery device of the present disclosure may comprise, e.g., without being limiting, one or more of the following or their equivalents, derivatives or analogs: thrombin inhibitors; antithrombogenic agents; thrombolytic agents; fibrinolytic agents; vasospasm inhibitors; vasodilators; antihypertensive agents; antimicrobial agents, such as Benzalkonium (BAK) or antibiotics (such as tetracycline, chlortetracycline, bacitracin, neomycin, polymyxin, gramicidin, cephalexin, oxytetracycline, chloramphenicol, rifampicin, ciprofloxacin, tobramycin, gentamycin, erythromycin, penicillin, sulfonamides, sulfadiazine, sulfacetamide, sulfamethizole, sulfisoxazole, nitrofurazone, sodium propionate), antifungals (such as amphotericin B and miconazole), and antivirals (such as idoxuridine trifluorothymidine, acyclovir, gancyclovir, interferon); inhibitors of surface glycoprotein receptors; antiplatelet agents; antimitotics; microtubule inhibitors; anti-secretory agents; active inhibitors; remodeling inhibitors; antisense nucleotides; anti-metabolites; antiproliferatives (including antiangiogenesis agents); anticancer chemotherapeutic agents; anti-inflammatories (such as hydrocortisone, hydrocortisone acetate, dexamethasone 21-phosphate, fluocinolone, medrysone, methylprednisolone, prednisolone 21-phosphate, prednisolone acetate, fluoromethalone, betamethasone, triamcinolone, triamcinolone acetonide); and non-steroidal anti-inflammatories (NSAIDs) (such as salicylate, indomethacin, ibuprofen, diclofenac, flurbiprofen, piroxicam indomethacin, ibuprofen, naxopren, piroxicam and nabumetone). Such anti-inflammatory steroids contemplated for use in the methodology of the embodiments described here, include triamcinolone acetonide (generic name) and corticosteroids that include, for example, triamcinolone, dexamethasone, fluocinolone, cortisone, prednisolone, flumetholone, and derivatives thereof); antiallergenics (such as sodium chromoglycate, antazoline, methapyriline, chlorpheniramine, cetrizine, pyrilamine, prophenpyridamine); anti proliferative agents (such as 1,3-cis retinoic acid, 5-fluorouracil, taxol, rapamycin, mitomycin C and cisplatin); decongestants (such as phenylephrine, naphazoline, tetrahydrazoline); miotics and anti-cholinesterase (such as pilocarpine, salicylate, carbachol, acetylcholine chloride, physostigmine, eserine, diisopropyl fluorophosphate, phospholine iodine, demecarium bromide); antineoplastics (such as carmustine, cisplatin, fluorouracil3; immunological drugs (such as vaccines and immune stimulants); hormonal agents (such as estrogens, -estradiol, progestational, progesterone, insulin, calcitonin, parathyroid hormone, peptide and vasopressin hypothalamus releasing factor); immunosuppressive agents, growth hormone antagonists, growth factors (such as epidermal growth factor, fibroblast growth factor, platelet derived growth factor, transforming growth factor beta, somatotrapin, fibronectin); inhibitors of angiogenesis (such as angiostatin, anecortave acetate, thrombospondin, anti-VEGF antibody); dopamine agonists; radiotherapeutic agents; peptides; proteins; enzymes; extracellular matrix; components; ACE inhibitors; free radical scavengers; chelators; antioxidants; anti polymerases; photodynamic therapy agents; gene therapy agents; and other therapeutic agents such as prostaglandins, antiprostaglandins, prostaglandin precursors, including antiglaucoma drugs including beta-blockers such as Timolol, betaxolol, levobunolol, atenolol, and additional prostaglandin analogues such as travoprost, latanoprost etc; carbonic anhydrase inhibitors such as acetazolamide, dorzolamide, brinzolamide, methazolamide, dichlorphenamide, diamox; and neuroprotectants such as lubezole, nimodipine and related compounds; and parasympathomimetrics such as pilocarpine, carbachol, physostigmine and the like.

The composition of this invention may also include one or more additives or excipients. For example, the composition may contain an inert filler material, a salt, a surfactant, a dispersant, a second polymer, a tonicity agent, or a combination thereof. See, e.g., U.S. Patent Application Publication 2009/0104243.

In some embodiments, additives and/or excipients in the ocular insert comprising a polymer matrix and stable amorphous bimatoprost includes a phospholipid (e.g., 1,2-dimyristoyl-sn-glycero-3-phosphocholine (DMPC)), stearyl alcohol, and/or carbopol.

In some embodiments, release rate modifying additives may be used to control the release kinetics of bimatoprost. For example, the additives may be used to control the concentration of bimatoprost by increasing or decreasing solubility of bimatoprost in the drug core so as to control the release kinetics. The solubility may be controlled by providing appropriate molecules and/or substances that increase and/or decrease the solubility of bimatoprost to the matrix. The solubility of bimatoprost may be related to the hydrophobic and/or hydrophilic properties of the matrix and therapeutic agent. For example, surfactants, tinuvin, salts and water can be added to the matrix and may increase the solubility of bimatoprost in the matrix. Salts can be water soluble, such as sodium chloride, or water-insoluble, such as titanium dioxide. In addition, oils and hydrophobic molecules and can be added to the matrix and may increase the solubility of hydrophobic treatment agent in the matrix. Alternatively, various oligomers and polymers, for example polysaccharides such as alginates, or proteins such as albumin, can be added. Solvents such as glycerol can also be used to modify the rate of release of the agent from the matrix into the tear liquid.

### Stability

In some embodiments, ocular inserts comprising bimatoprost and thermoplastic polymer, as described under the “Compositions” section of the current disclosure, have a shelf life or stability of about 18 months to about 36 months or more. The stability is measured after storing the ocular inserts of the current disclosure in a humidity chamber with a relative humidity (RH) of about 60% to about 70%, and temperature of about 25° C.±2° C. to about 40° C.±2° C. For example, the accelerated stability data is measured at about 40° C.±2° C. under about 75% relative humidity (RH). Accelerated aging parameters, including information that validates the accelerated system are required for product shelf-life testing. Real time testing of shelf life is also performed in order to confirm the tentative shelf life data collected from the accelerated tests. In addition, the shelf life of ocular inserts is also tested under expected packaging conditions, for example when the ocular inserts are packaged in the presence of an oxygen absorber. Tensile strength is also tested under accelerated conditions (high temperature and relative humidity).

To test stability, samples are withdrawn at 0, 1, 2, 3, 4, 5, and 6, or more months. In some embodiments, shelf life value of ocular insert comprising composition of bimatoprost and silicone is about 6 months, about 7 months, about 8 months, about 9 months, about 10 months, about 11 months, about 12 months, about 13 months, about 14 months, about 15 months, about 16 months, about 17 months, about 18 months, about 19 months, about 20 months, about 21 months, about 22 months, about 23 months, about 24 months, about 25 months, about 26 months, 27 months, about 28 months, about 29 months, about 30 months, about 31 months, about 32 months, about 33 months, about 34 months, about 35 months, about 36 months, about 37 months, about 38 months, about 39 months, about 40 months, about 41 months, about 42 months, about 43 months, about 44 months, about 45 months, or more. In some embodiments, the accelerated tensile strength (aged at about 55° C.) of the ocular inserts comprising bimatoprost and a thermoplastic polymer is about 1.9, about 2.0, about 2.1, about 2.2, about 2.3, about 2.4, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, or about 4.5. In one embodiment, the average accelerated tensile strength (aged at about 55° C.) of ocular inserts comprising bimatoprost and a thermoplastic polymer is 2.9±0.6.

### Process of Preparing the Compositions

Bimatoprost solid is dissolved in an organic solvent and the solution is then mixed with polymer, e.g., silicone. The amounts of bimatoprost and polymer are predetermined to ensure a therapeutically sufficient amount of bimatoprost is released into the body fluid. For example, the weight of bimatoprost is about 0.1 to about 40%, about 1% to about 30%, about 5% to about 30%, about 5% to about 25%, or about 5% to about 22% of the total weight of bimatoprost and polymer. Organic solvents that can be used here are preferably those that easily dissolve bimatoprost and have low boiling points so that they can be easily removed. Examples of suitable organic solvents include, but are not limited to, dichloromethane, chloroform, ether (e.g., diethyl ether), esters (e.g., ethyl acetate), acetonitrile, or acetone. The polymer can be a thermosetting polymer or thermoplastic polymer. For an illustrative purpose, preparation of compositions containing bimatoprost and silicone will be described below. Other polymers can be used in a similar manner to prepare the compositions within the scope of this invention. Examples of suitable silicones include, but are not limited to, those commercially available from NuSil Technology or Polymer Systems Technology, Ltd. under catalog numbers of the MED-4800 series (e.g. MED-4810, MED-4820, MED-4830, MED-4840, MED-4842, MED1-4855, MED-4860, MED-4870, or MED-4880).

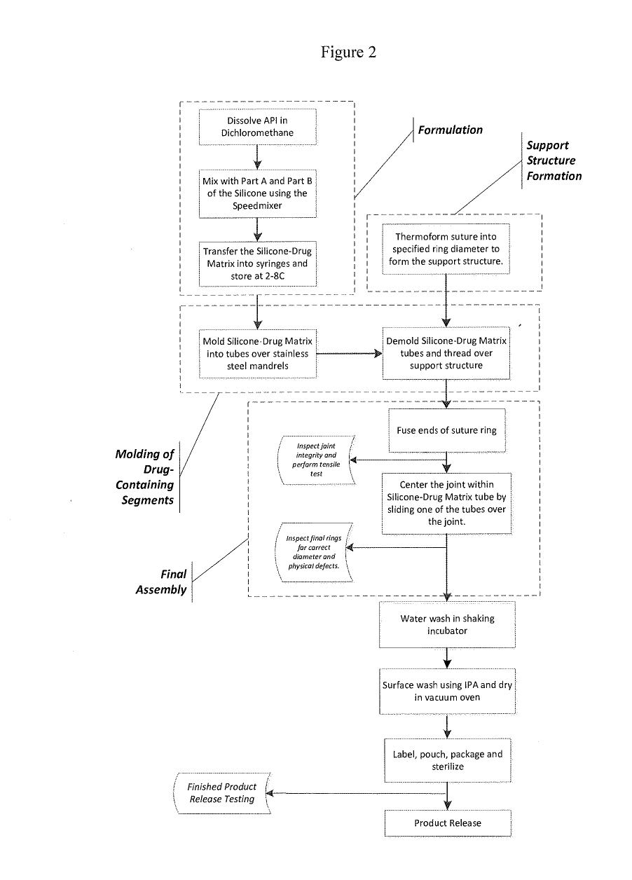

The solvent is removed by a conventional method, e.g., under reduced pressure, and the mixture of bimatoprost and silicone is molded into a pre-designed shape. In one embodiment, the mixture is pressure-injected a tube mold with a stainless steel mandrel and the obtained tube is threaded over a support structure of a ring shape having a predetermined diameter. See FIG. 2.

During or after the molding, the bimatoprost and silicone mixture is heated to cure the silicone. The heating temperature is set to be higher than the melting point of bimatoprost, for example, equal to or above about 65° C., equal to or above about 80° C., equal to or above about 100° C., equal to or above about 140° C. to about 160° C., or about 152° C. The curing process may last for 2 to 10 minutes at the elevated temperature (e.g., about 5 minutes at about 152° C.). The bimatoprost co-mixed with silicone is melted during the curing process. Upon cooling, the bimatoprost solidifies into a stable amorphous form while the silicone is cured to form a silicone matrix. Surprisingly, the bimatoprost remains amorphous after long term storage (e.g., 6 months or even longer at 40° C. and 75% relative humidity). See, e.g., FIG. 2.

The resulting device can be washed with water or an organic solvent and sterilized by, e.g., e-beam, before its therapeutic application to a patient. See, e.g., FIG. 2. The organic solvent used for the wash can be selected from dichloromethane, chloroform, acetone, acetonitrile, methanol, ethanol, isopropanol, ethyl acetate, diethyl ether, and a mixture thereof.

In another embodiment a composition of the ocular insert of the present disclosure is prepared by a process comprising heating bimatoprost at a temperature above its crystal melting temperature (e.g., at about 65° C., about 70° C., about 75° C., or about 80° C.) to produce a bimatoprost melt; mixing the melt with an uncured polymer at a ratio of about 2%, about 5%, about 7%, about 10%, about 20%, about 25%, or about 30%, in one or more mixing cycles for about 5 minutes, about 10 minutes, about 20 minutes, about 30 minutes, or about 1 hour each; and curing the polymer at a temperature above the melting point of bimatoprost, e.g., at about 60° C., about 100° C., about 140° C., about 150° C., or about 160° C., for about 3 minutes, about 5 minutes, or about 7 minutes. In one embodiment, a composition of the ocular insert is prepared by a process comprising heating bimatoprost, e.g., at about 70° C. to form a melt, mixing the melt with an uncured polymer at a ratio of about 20% in one or more mixing cycles for about 30 minutes each, and curing the polymer, e.g., at about 150° C., the temperature above the melting point of bimatoprost, for about 5 minutes.

In yet another embodiment, a composition of the ocular insert of the present disclosure is prepared by a process comprising mixing bimatoprost powder with an uncured polymer at a ratio of about 2%, about 5%, about 7%, about 10%, about 20%, about 25%, or about 30%, in one or more cycles for about 1 minute, about 5 minutes, about 10 minutes, about 20 minutes, about 30 minutes, or about 1 hour each; heating the mixture, while mixing, at a temperature above the bimatoprost crystal melting temperature to melt bimatoprost into the polymer, e.g., at about 65° C., about 70° C., about 75° C., or about 80° C., in one or more mixing cycles for about 5 minutes, about 10 minutes, about 20 minutes, about 30 minutes, or about 1 hour each; and curing the polymer at a temperature above the melting point of bimatoprost, e.g., at about 60° C., about 100° C., about 140° C., about 150° C., or about 160° C., for about 3 minutes, about 5 minutes, or about 7 minutes. In one embodiment, a composition of the ocular insert is prepared by a process comprising mixing bimatoprost powder with an uncured polymer at a ratio of about 20% in one or more cycles for about 30 minutes each; while mixing, heating the mixture at a temperature above the bimatoprost crystal melting temperature to melt the bimatoprost into the polymer, e.g., at about 70° C., in one or more cycles for about 30 minutes each; curing the polymer at a temperature above the melting point of bimatoprost, e.g., at about 150° C., for about 5 minutes.

Methods of Use in the Treatment, Prevention, and/or Alleviation of an Ocular Disease and/or Disorder

Compositions of this invention can be prepared as a device, e.g., a medical device, such as an ocular device. An ocular device can be used to treat eye disease.

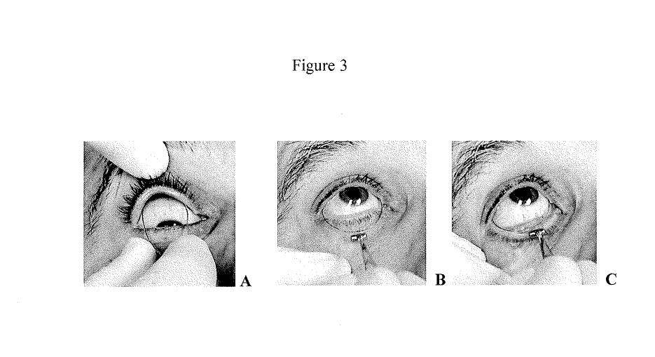

The device having a ring shape as prepared above can be placed on or in an eye to reduce intraocular pressure. For example, following administration of a drop of anesthetic agent, the eyelids are gently spread open and, using a blunt-ended surgical instrument, the ocular insert is placed in the upper and lower fornices, as shown in FIG. 3. The ocular device may be kept in place for a long period of time, during which time bimatoprost is continuously released to the eye at a therapeutically effective level so as to exert the sustained reduction of intraocular pressure. Such reduction in IOP can thereby treat or alleviate a symptom of glaucoma.

Although it is not intended to be a limitation of the invention, it is believed bimatoprost transports through the silicone matrix to its surface whereupon the agent becomes dispersed, dissolved or otherwise entrained with body fluid, e.g., tear liquid. The transport may be the result of and/or influenced by diffusion, molecular interaction, domain formation and transport, infusion of body fluid into the matrix or other mechanisms. For delivery to the eye, a therapeutically effective amount of bimatoprost transports to the exposed surface of the matrix whereupon tear liquid will sweep away the agent for delivery to target tissue or tissues.

Surprisingly, the amorphous bimatoprost in the composition of this invention retains the amorphous structure even after an extended period of time of elution, e.g., 6 months, 9 months, 18 months, 36 months or more.

### DEFINITIONS

As used herein, the term “bimatoprost” refers to 7-[3,5-dihydroxy-2-(3-hydroxy-5-phenyl-pent-1-enyl)-cyclopentyl]-N-ethyl-hept-5-enamide:

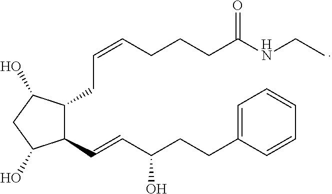 [SMILES: CCNC(=O)CCC/C=C\C[C@@H]1[C@@H](/C=C/[C@@H](O)CCc2ccccc2)[C@H](O)C[C@@H]1O]

Bimatoprost is marketed by Allergan as an ophthalmic solution called LUMIGAN®. It is also sold as a cosmetic formulation known as LATISSE®. The synthesis and purification of bimatoprost is described, e.g., in U.S. Pat. No. 7,157,590.

As used herein, the terms “cure,” “curing,” and “cured” refer to the toughening or hardening of a polymer material by cross-linking of polymer chains, brought about by chemical additives, ultraviolet radiation, electron beam or heat. In one aspect, the polymer is silicone.

As used herein, the term “process,” “processing,” and “processed” refer to reforming intermolecular interactions to remold thermoplastics. Processing is usually achieved by heating and cooling thermoplastics.

As used herein, the term “silicone” refers to polysiloxanes. In one aspect, the silicone has two parts or components, e.g., Part A and Part B, component A or component B. For example, Part A (or component A) may comprise of silica (e.g., about 20% silica). Part B (or component B) may comprise of silica (e.g., about 20% silica) and poly(dimethylsiloxane-co-methylhydrosiloxane) (e.g., less than about 3% and where the poly(dimethylsiloxane-co-methylhydrosiloxane) is trimethylsilyl terminated). In another aspect, silicone may be purchased from NuSil Technology or Polymer Systems Technology, Ltd. under a catalog number of the MED-4800 series (e.g. MED-4810, MED-4810 Part A, MED-4810 Part B, MED-4820, MED-4830, MED-4840, MED-4842, MED1-4855, MED-4860, MED-4870, or MED-4880).

As used herein, the term “medical device” refers to a drug-delivery system or device that affects or controls the release and/or delivery of the therapeutic agent in a certain way(s).

As used herein, the terms “ocular insert” and “ocular device” refer to a bimatoprost-impregnated device, whose size and shape are designed for ophthalmic application. See, e.g., Kumari A. et al., J. Adv. Pharm. Technol. Res. 2010, 1(3): 291-296. In one aspect, the insert may be sterile, thin, multilayered, drug-impregnated, solid or semisolid consistency. In another aspect, the insert may be placed into the cul-de-sac or conjunctival sac. Manufacturing and administration of various ocular inserts have been described in the literature. See, e.g., Kumari A. et al. J. Adv. Pharm. Technol. Res. 2010, 1(3): 291-296. In one aspect, the insert or device may be sterile, thin, multilayered, drug-impregnated, solid or semisolid consistency. In another aspect, the insert may be placed into the cul-de-sac or conjunctival sac.

As used herein “crystalline” means that the compound is crystallized into a specific crystal packing arrangement in three spatial dimensions or the compound having external face planes. Compounds in the crystalline state exhibit distinct sharp peaks in their X-ray diffraction patterns and typically exhibit well defined melting points. For example, bimatoprost can crystallize into different crystal packing arrangements, all of which have the same elemental composition of bimatoprost. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystals of bimatoprost may be prepared by crystallization under different conditions, e.g., different solvents, temperatures, etc.

As used herein “amorphous” or “non-crystalline” means that the compound does not exhibit any substantial peaks in its X-ray diffraction pattern. Typically, non-crystalline materials do not exhibit well defined melting points.

As used herein, the term “stable amorphous” means that the compound is capable of retaining the amorphous form for more than 1 day, 30 days, 60 days, 90 days, 120 days, 150 days, 180 days, 6 months, 9 months, 18 months, 36 months or more.

All percentages and ratios used herein, unless otherwise indicated, are by weight.

Although specific reference is made to a ring-shaped ocular insert, medical devices or apparatus having different features can be prepared and used according to the known methods. Such embodiments are within the scope of this invention. For example, U.S. Ser. No. 13/618,052, U.S. Ser. No. 13/688,019, and WO2013/040426, specifically incorporated by reference herein, describe many embodiments of an ocular insert that can be comfortably placed at many locations of the conjunctiva, including along at least a portion of the conjunctival sac. The insert can move when placed on the conjunctiva and can be retained with the eye so as to provide improved comfort for the patient. The insert may comprise a resistance to deflection to retain the insert comfortably within the eye. The insert can be configured in many ways to provide the resistance to deflection. The insert may comprise a matrix comprising the resistance to deflection, and the matrix may comprise a material providing the resistance to deflection. Alternatively or in combination, the insert may comprise a retention structure and a support structure coupled to the retention structure, in which the support structure may contain the therapeutic agent. The retention structure may comprise an inner structure with the support structure comprising the therapeutic agent covering at least a portion of the retention structure, or the retention structure may comprise an outer structure covering at least a portion of the support structure comprising the therapeutic agent.

The insert may be configured such that the insert can be deflected during insertion and removal and may comprise the resistance to deflection for comfort and retention. The insert comprising the resistance to deflection can be comfortably placed at one or more of many locations of the conjunctiva, such that many patients can be treated comfortably and the placement can be adjusted based on the anatomy of the patient and physician preference. The insert may comprise the resistance to deflection such that the conjunctiva can be shaped with the insert so as to receive the insert, and in many embodiments the insert may comprise an amount of resistance to form one or more of a fold, a pocket, or deformation of the conjunctiva so as to receive and retain the insert. The one or more locations where the insert can be placed include the inferior conjunctival sac, an inferior temporal location of the conjunctival sac, an inferior nasal location of the conjunctival sac, the superior conjunctival sac, portions of the upper and lower conjunctival sacs near lateral canthus of the palpebral fissure, portions of the upper and lower conjunctival sacs near the medial canthus and caruncle. These areas are well suited to receive structures having relatively large volumes for extended release of one or more therapeutic agents. In one embodiment, the ocular insert is positioned on a region outside an optical zone of an eye.

The insert can be configured in many ways to treat a patient with bimatoprost for an extended time, and may comprise one or more of a high dose of therapeutic agent, a substantial surface area to release the therapeutic agent, a hoop strength to resist deflection, a bending strength to resist deflection, a shape profile to fit the eye, or a biasing curve to retain the insert, and combinations thereof. The insert may comprise biasing shape so as to retain the insert, for example with a curve, bend, or other deflected shape to retain the insert. The biasing shape may comprise a resiliently curved biasing spring structure shaped to provide force in response to deflection so as to urge one or more of the first portion or the second portion toward the eye to retain the insert.

The insert can be sized and shaped for placement under the eyelids and along at least a portion of a conjunctival sac of the upper and lower lids of the eye, or combinations thereof. The insert can be sized and shaped so as to move within the conjunctival sac of the eye and be held on the eye without attachment to the eye so as to provide improved comfort. The insert may comprise a preformed shape profile corresponding to a curved shape profile of the eye extending away from a plane, such that the insert can resist deflection away from bulbar conjunctiva toward the plane when placed. The insert can be configured to deflect when placed in the conjunctival sac of the eye and guide the insert along the sac when the eye moves with one or more of rotation or cyclotorsion. The insert may also comprise resistance to deflection so as to urge the insert outward and inhibit movement of the retention structure toward the cornea. The insert may comprise a first portion having a first resistance to deflection and a second portion having a second resistance to deflection less than the first portion, such that first portion can resist deflection of the upper lid and the second portion can fit within the one or more folds of the lower lid. The first portion and the second portion may comprise a similar material, and the first portion may have a cross sectional size greater than the second portion to provide the increased resistance to deflection, and the increased cross sectional size of the first portion may help to retain the first portion with the upper lid. Alternatively or in combination, the increased cross-sectional size of the first portion may provide anchoring under the upper lid. The insert may move rotationally with deflection along the conjunctival sac such that the retention structure can slide along the conjunctival sac about an axis of rotation passing through the iris and the pupil of the eye. In many embodiments the insert can allow sliding movement along the conjunctiva in response to torsional or other movement of the eye so as to improve comfort for the patient.

The insert can be configured in many ways to provide the resistance to deflection. The insert may comprise a retention structure providing a majority of the resistance to deflection. Alternatively, the insert can be configured to provide the resistance to deflection without a retention structure, and in many embodiments may comprise with a drug delivery matrix configured to provide the resistance to deflection such that the insert can be provided without the retention structure.

The eye comprises upper and lower conjunctival sacs corresponding to the upper eyelid and the lower eyelid, and each of the upper and lower conjunctival sacs comprises a bulbar portion of conjunctiva and a palpebral portion of conjunctiva. The bulbar portion and the palpebral portion of each sac may comprise a plurality of folds, and the insert may comprise a resistance to deflection so as to shape the conjunctiva and form one or more of an indentation, a deformation, a fold or a pocket of the conjunctiva. The insert can be elongate and sized to extend a substantial distance along the shaped conjunctiva, such that the retention structure can be held with the one or more of the indentation, the deformation, the fold or the pocket of the conjunctiva. The palpebral and bulbar conjunctiva may each be shaped with the retention structure so as to comprise one or more folds or pockets, and the insert can extend substantially along the one or more folds or pockets such that the retention structure can move with the eye. The shaped conjunctival tissue may comprise tissue of the fornix, or conjunctival tissue located away from the fornix, or combinations thereof. The movement of the insert along the conjunctival sac, resistance to inward deflection, resistance to deflection to shape the conjunctiva can provide improved comfort for the patient.

The retention structure can be configured in many ways to provide increased comfort for the patient, and can be placed in many ways. The retention structure may comprise soft material at locations corresponding to one or more of the lacrimal gland or the caruncle, and can be shaped to inhibit contact with tissue near one or more of the lacrimal gland or the caruncle. Although the retention structure may comprise one or more of many shapes such as circular, oval, serpentine, saddle shaped, cylindrical or toric, the retention structure may comprise one or more portions shaped to inhibit irritation to the lacrimal gland and the caruncle. The retention structure can be shaped to inhibit contact with the conjunctiva covering the lacrimal gland, and the retention structure may comprise an extension shaped to extend around the lacrimal gland. The extension can extend inward toward the pupil around the lacrimal gland, or outward away from the pupil around the lacrimal gland. The retention structure may comprise a portion shaped to extend away from the caruncle when placed, such as an inward extension.

“Treating”, includes any effect, e.g., lessening, reducing, modulating, or eliminating, that results in the improvement of the condition, disease, disorder, etc. “Treating” or “treatment” of a disease state includes: (1) inhibiting the disease state, i.e., arresting the development of the disease state or its clinical symptoms; (2) relieving the disease state, i.e., causing temporary or permanent regression of the disease state or its clinical symptoms; or (3) reducing or lessening the symptoms of the disease state.

“Preventing” includes any effect in, e.g., causing the clinical symptoms of the disease state not to develop in a subject that may be exposed to or predisposed to the disease state, but does not yet experience or display symptoms of the disease state. As used herein, “preventing” or “prevent” describes reducing or eliminating the onset of the symptoms or complications of the disease, condition or disorder. The term “preventing,” when used in relation to a condition, such as intraocular pressure, is art-recognized, and refers to formulation, composition and/or device (e.g., ocular insert) which reduces the frequency of, or delays the onset of, signs and/or symptoms of a medical condition in a subject relative to a subject which does not receive the composition.

As used herein, the term “alleviate” is meant to describe a process by which the severity of a sign or symptom of a disorder is decreased. Importantly, a sign or symptom can be alleviated without being eliminated.

As used herein the term “symptom” is defined as an indication of disease, illness, injury, or that something is not right in the body. Symptoms are felt or noticed by the individual who is experiencing the symptom, but may not easily be noticed by others. Others are defined as non-health-care professionals.

As used herein the term “sign” is also defined as an indication that something is not right in the body. But signs are defined as things that can be seen by a doctor, nurse, or other health care professional.

The term “about” is used herein to mean approximately, in the region of, roughly or around. When the term “about” is used in conjunction with a numerical range, it modifies that range by extending the boundaries above and below the numerical values set forth. In general, the term “about” is used herein to modify a numerical value above and below the stated value by a variance of 20%.

As used in the present disclosure, whether in a transitional phrase or in the body of a claim, the terms “comprise(s)” and “comprising” are to be interpreted as having an open-ended meaning That is, the terms are to be interpreted synonymously with the phrases “having at least” or “including at least.” When used in the context of a process the term “comprising” means that the process includes at least the recited steps, but may include additional steps. When used in the context of a molecule, compound, or composition, the term “comprising” means that the compound or composition includes at least the recited features or components, but may also include additional features or components.

For the purposes of promoting an understanding of the embodiments described herein, reference made to preferred embodiments and specific language are used to describe the same. The terminology used herein is for the purpose of describing particular embodiments only, and is not intended to limit the scope of the present invention. As used throughout this disclosure, the singular forms “a,” “an,” and “the” include plural reference unless the context clearly dictates otherwise. Thus, for example, a reference to “a composition” includes a plurality of such compositions, as well as a single composition, and a reference to “a therapeutic agent” is a reference to one or more therapeutic and/or pharmaceutical agents and equivalents thereof known to those skilled in the art, and so forth. All percentages and ratios used herein, unless otherwise indicated, are by weight.

The term “more” as used in the present disclosure does not include infinite number of possibilities. The term “more” as used in the present disclosure is used as a skilled person in the art would understand in the context in which it is used. For example, more than “36 months” implies, as a skilled artisan would understand, 37 months or the number of months the ocular insert can be or is used by a subject, which is greater than 36 months, without loss of efficacy of the therapeutic agent in the insert.

Unless otherwise defined, all technical and scientific terms used herein have the same meaning as commonly understood by one of ordinary skill in the art to which this invention belongs. In the case of conflict, the present specification will control. In the specification, the singular forms also include the plural unless the context clearly dictates otherwise. Although methods and materials similar or equivalent to those described herein can be used in the practice or testing of the present invention, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting.

### EXAMPLES

### Example 1

### Preparation of Compositions

A composition of bimatoprost and silicone was prepared by dissolving bimatoprost in dichloromethane, mixing the resulting composition with Part A and Part B of silicone MED 4810. Dichloromethane was removed under vacuum.

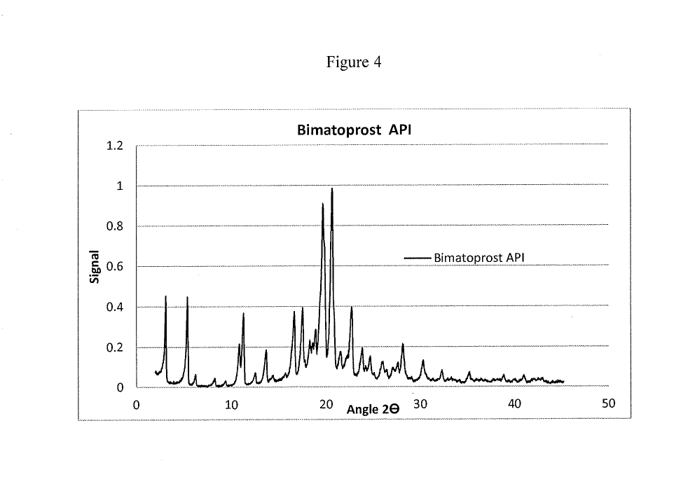

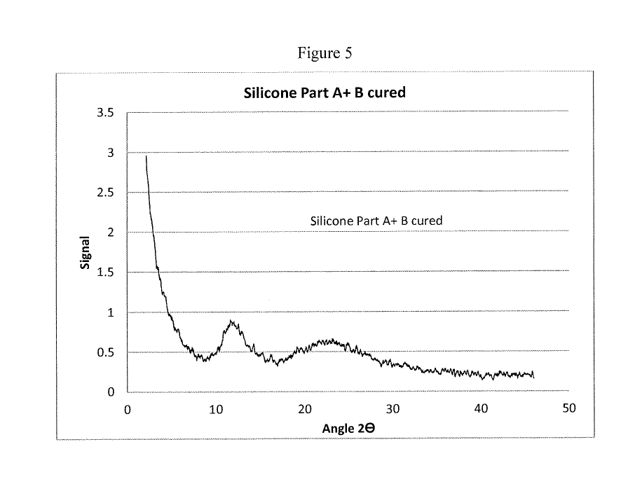

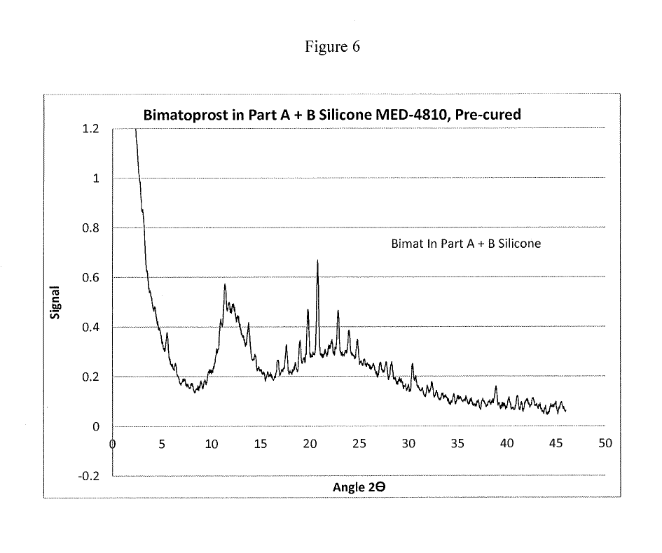

FIG. 4 shows the characteristic peaks for commercially available bimatoprost crystalline solid. FIG. 5 shows an X-ray powder diffraction pattern of cured Part A and Part B of MED-4810 silicone, which does not contain any sharp peak. FIG. 6 is an X-ray powder diffraction pattern of 7% of bimatoprost in Part A and Part B of MED-4810 silicone before curing. The pattern contains sharp peaks for bimatoprost, which indicates that there was crystalline bimatoprost.

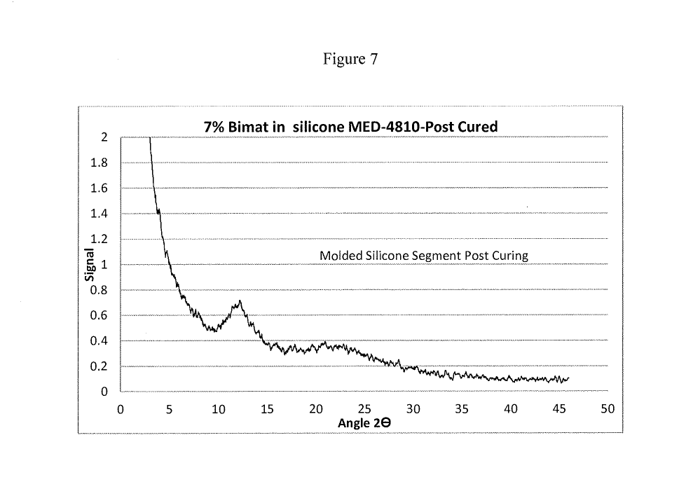

After removal of dichloromethane, the resulting composition was cured at about 305° F. (about 152° C.) for about 5 minutes. FIG. 7 is an X-ray powder diffraction pattern of the thus-cured composition. The pattern does not have any peaks, indicating that the composition is non-crystalline.

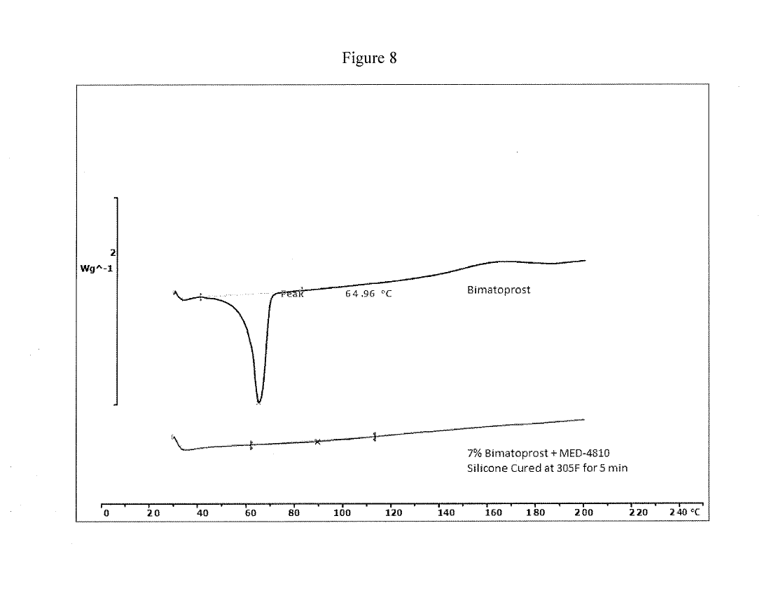

FIG. 8 shows the differential scanning calorimetry profiles of bimatoprost and of 7% bimatoprost in 305° F. cured Part A and Part B of MED-4810 at 10° C./min under nitrogen. The melting point of bimatoprost alone is at about 64.9° C. or at about 64.96° C. This indicates that that the sample is highly crystalline, and the presence of a single melting peak suggests only one crystalline form of bimatoprost. The profile of the 7% bimatoprost in cured Part A and Part B of MED-4810 silicone cured at 305° F. for 5 minutes has no obvious melting peak, indicating that the material is non-crystalline.

Additionally, a composition of bimatoprost and MED-4830 was prepared in a similar manner as described above. The obtained product was immersed in water (e.g. deionized water, tap water, water for injection) at approximately 60±10° C. for typically 48-72 hours and then was sterilized with electron beam irradiation at a minimal effective dose of 17.5 kGy (e.g. Nutek Corporation E-Beam System 4).

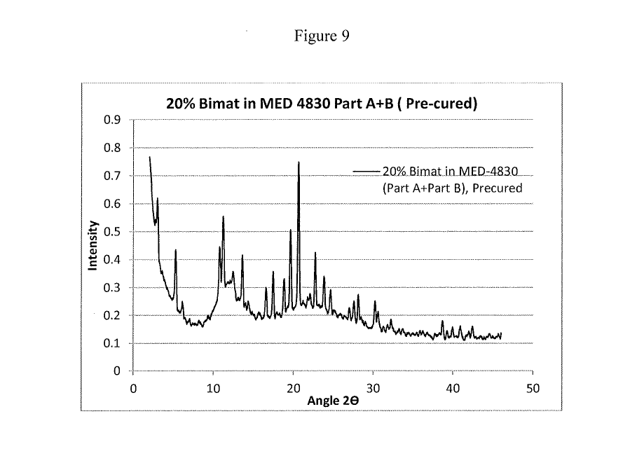

FIG. 9 is an X-ray powder diffraction pattern of 20% of bimatoprost in Part A and Part B of MED-4830 silicone before curing. The pattern shows distinct sharp peaks, indicating that the bimatoprost is crystalline.

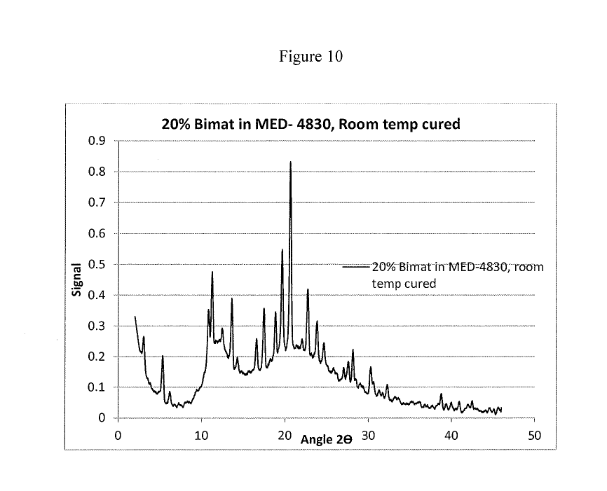

FIG. 10 is an X-ray powder diffraction pattern of 20% of bimatoprost in Part A and Part B of MED-4810 silicone cured at room temperature. The pattern shows distinct sharp peaks for bimatoprost, suggesting that curing silicone with bimatoprost at room temperature does not destroy the crystalline structure of bimatoprost in the composition.

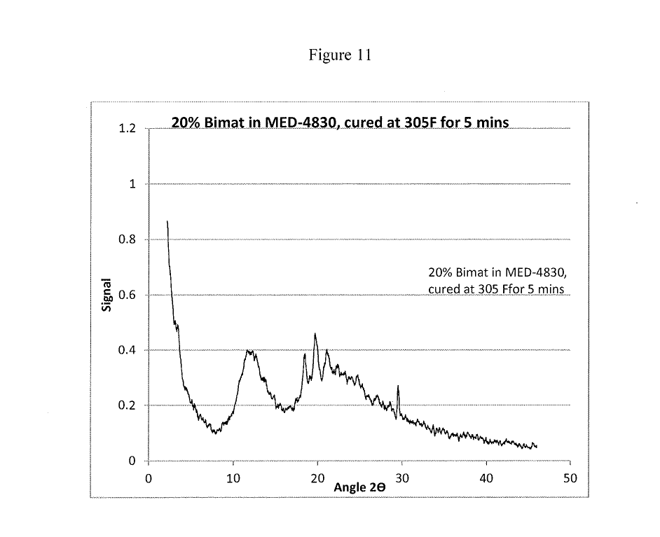

FIG. 11 is an X-ray powder diffraction pattern of 20% of bimatoprost in Part A and Part B of MED-4830 silicone cured at 305° F. for 5 minutes, prior to washing and irradiation by electron beam. The pattern shows some distinct peaks, suggesting that the high temperature cured composition contains some crystalline bimatoprost.

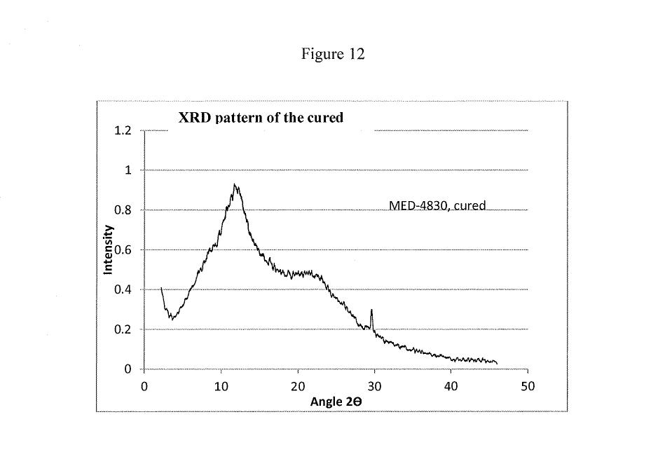

FIG. 12 is an X-ray powder diffraction pattern of a cured MED-4830 without bimatoprost. It demonstrates the non-crystalline nature of the cured silicone material.

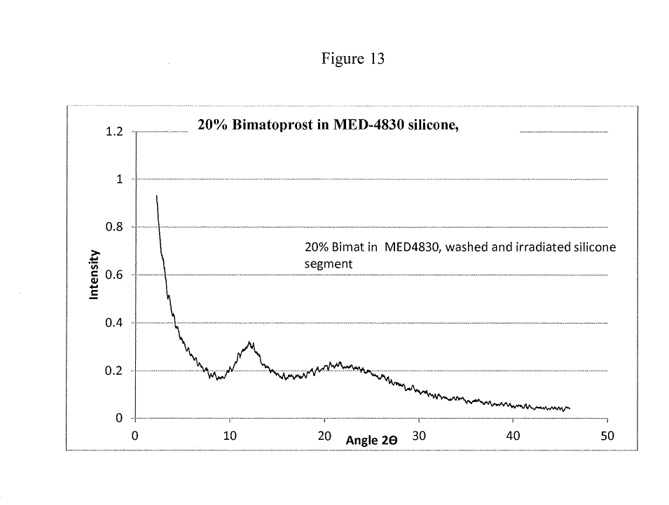

FIG. 13 is an X-ray powder diffraction pattern of 20% of bimatoprost in cured MED-4830 silicone, after being washed and irradiated by electron beam. The pattern does not have any peaks indicating that the composition is non-crystalline. This, in conjunction with FIG. 11, suggests that bimatoprost that is on the surface of the silicone crystallizes upon cooling and that this surface material is removed during the washing and irradiation steps, but that bimatoprost within the silicone matrix remains non-crystalline.

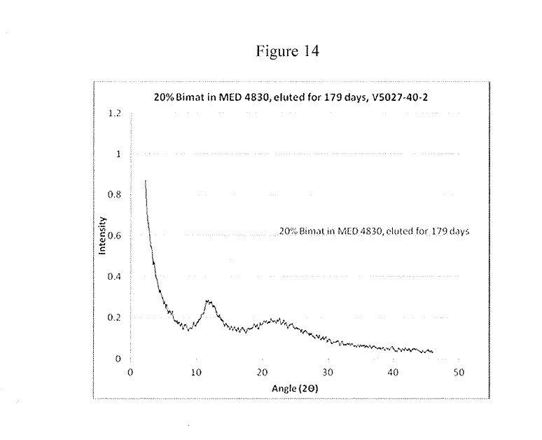

A composition of bimatoprost and silicone was also prepared by dissolving bimatoprost in dichloromethane, mixing the resulting composition with Part A and Part B of silicone MED 4830. Dichloromethane was removed. After dichloromethane was removed, the resulting composition was cured at about 305° F. for about 5 minutes. The resulting composition was washed in water at 60 C for 48-72 hours, rinsed in 70% isopropanol, dried, and packaged. The product was eluted in 0.5% sodium dodecyl sulfate/phosphate buffer solution for 179 days at 37° C. The X-ray powder diffraction pattern (see FIG. 14) does not show any distinct peak, indicating that the bimatoprost did not re-crystallize during the time of elution.

### Example 2

### Stability Studies

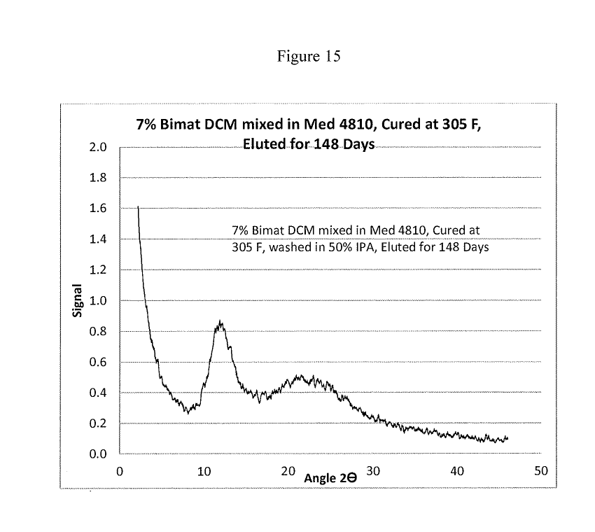

The stability of the compositions of the invention was carried out using methods known in the art. A composition of bimatoprost and silicone was prepared by dissolving bimatoprost in dichloromethane, mixing the resulting composition with Part A and Part B of silicone MED 4810. Dichloromethane was removed. After dichloromethane was removed, the resulting composition was cured at about 305° F. for about 5 minutes. The resulting composition was washed in 50% isopropanol and eluted in 0.5% sodium dodecyl sulfate/phosphate buffer solution for 148 days at 37° C. The X-ray powder diffraction pattern (see FIG. 15) is similar to that of FIG. 7, indicating that the bimatoprost did not re-crystallize during the time of elution.

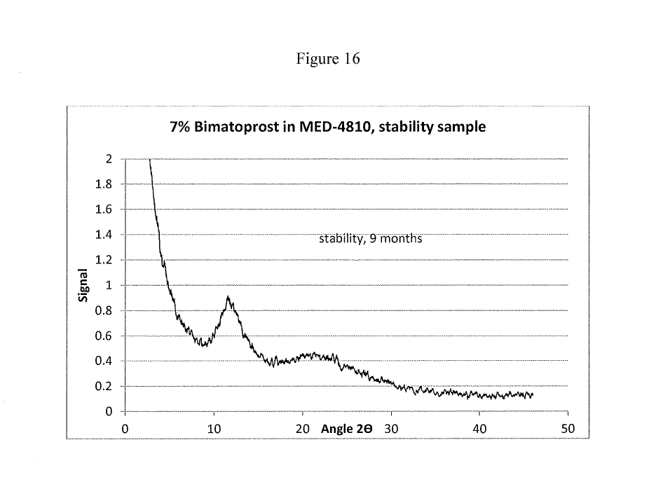

A composition of bimatoprost and silicone was also prepared by dissolving bimatoprost in dichloromethane, mixing the resulting composition with Part A and Part B of silicone MED 4810. Dichloromethane was removed. After dichloromethane was removed, the resulting composition was cured at about 305° F. for about 5 minutes. The resulting composition was washed in 50% isopropanol and stored for 9 months at 40° C. and 75% relative humidity. The X-ray powder diffraction pattern (see FIG. 16) is similar to those of FIG. 7 and FIG. 15, indicating the bimatoprost did not re-crystallize during the time of storage.

Stability studies were carried out on ocular inserts comprising composition of bimatoprost and silicone according to International Conference on Harmonization (ICH) guidelines. Ocular inserts were stored in a humidity chamber with a relative humidity (RH) of 60%-70%, and temperature of 25° C.±2° C. to 40° C.±2° C. For testing tensile strength under accelerated conditions (high temperature and relative humidity), the storage temperature was 55° C.±2° C.

Samples were withdrawn at 0, 1, 2, 3, 4, 5, and 6 months. Accelerated studies at elevated temperature and humidity revealed that shelf life value of ocular insert comprising composition of bimatoprost and silicone is about 18 months. However, as summarized in Tables 1-5, the shelf-life of the ocular insert comprising composition of bimatoprost and silicone can be 36 months or more.

The stability data two samples of the ocular insert drug product are summarized in Tables 1A, 1B, and 2A below.

TABLE 1A
Accelerated Stability Data (at high temperature and humidity,
40° C./75% RH) for Sample 1

| Time | Assay | 5-Trans | 15-Keto | RRT 0.59 | RRT 1.05 | Total Imp |
|---|---|---|---|---|---|---|
| (Month) | (%) | (%)* | (%)* | (%) | (%) | (%) |
| 3 | 99 | 0.2 | 0.4 | ND | ND | 0.6 |

*% Peak Area corrected for UV Response Factors: 1.06 for 5-Trans and 1.17 for 15-Keto

TABLE 1B
Real-Time Stability Data (25° C./60% RH) for Sample 1

| Time | Assay | 5-Trans | 15-Keto | RRT 0.57 | RRT 1.05 | Total Imp |
|---|---|---|---|---|---|---|
| (Month) | (%) | (%)* | (%)* | (%) | (%) | (%) |
| 0 | 101 | 0.2 | 0.4 | ND | ND | 0.6 |

*% Peak Area corrected for UV Response Factors: 1.06 for 5-Trans and 1.17 for 15-Keto

TABLE 2A
Real-Time Stability Data (25° C./60% RH) for Sample 2

| Time | Assay | 5-Trans | 15-Keto | RRT 0.57 | RRT 1.05 | Total Imp |
|---|---|---|---|---|---|---|
| (Month) | (%) | (%)* | (%)* | (%) | (%) | (%) |
| 0 | 100 | 0.2 | 0.4 | ND | ND | 0.6 |

*% Peak Area corrected for UV Response Factors: 1.06 for 5-Trans and 1.17 for 15-Keto

In addition, stability data for a research sample of the ocular insert (both 3-day and 10-day hold samples) is summarized in Tables 3A, 3B, 4A, and 4B below:

TABLE 3A
Accelerated Stability Data (at high temperature and humidity,
40° C./75% RH) for R&D Sample (3-day hold)

| Time | Assay | 5-Trans | 15-Keto | RRT 0.57 | RRT 1.05 | Total Imp |
|---|---|---|---|---|---|---|
| (Month) | (%) | (%)* | (%)* | (%) | (%) | (%) |
| 3 | 99 | 0.2 | 0.4 | ND | ND | 0.6 |
| 6 | 101 | 0.2 | 0.5 | ND | ND | 0.9 |

*% Peak Area corrected for UV Response Factors: 1.06 for 5-Trans and 1.17 for 15-Keto

TABLE 3B
Real-Time Stability Data (25° C./60% RH) for R&D Sample (3-day hold)

| Time | Assay | 5-Trans | 15-Keto | RRT 0.42 | RRT 1.05 | Total Imp |
|---|---|---|---|---|---|---|
| (Month) | (%) | (%)* | (%)* | (%) | (%) | (%) |
| 0 | 87 | 0.2 | 0.4 | ND | ND | 0.6 |
| 1 | 84 | 0.4 | 0.5 | 0.2 | ND | 1.2 |

*% Peak Area corrected for UV Response Factors: 1.06 for 5-Trans and 1.17 for 15-Keto

TABLE 4A
Accelerated Stability Data (at high temperature and humidity,
40° C./75% RH) for 10-day hold sample

| Time | Assay | 5-Trans | 15-Keto | RRT 0.76 | RRT 1.05 | Total Imp |
|---|---|---|---|---|---|---|
| (Month) | (%) | (%)* | (%)* | (%) | (%) | (%) |
| 3 | 99 | 0.2 | 0.5 | ND | ND | 0.7 |
| 6 | 104 | 0.2 | 0.5 | 0.1 | ND | 0.8 |

*% Peak Area corrected for UV Response Factors: 1.06 for 5-Trans and 1.17 for 15-Keto

TABLE 4B
Real-Time Stability Data (25° C./60% RH) for 10-day hold sample

| Time | Assay | 5-Trans | 15-Keto | RRT 0.57 | RRT 0.59 | Total Imp |
|---|---|---|---|---|---|---|
| (Month) | (%) | (%)* | (%)* | (%) | (%) | (%) |
| 0 | 89 | 0.1 | 0.4 | ND | 0.1 | 0.6 |
| 1 | 88 | 0.2 | 0.4 | 0.1 | ND | 0.7 |

*% Peak Area corrected for UV Response Factors: 1.06 for 5-Trans and 1.17 for 15-Keto

In addition to this data, real-time and accelerated stability was evaluated for products packaged with oxygen absorber. The data is summarized in Tables 5A and 5B.

TABLE 5A
Real-Time Stability Data (25° C.) for Ocular Inserts Packages
with Oxygen Absorber (3-day hold)

| Time | Assay | 5-Trans | 15-Keto | RRT 0.61 | RRT 1.05 | Total Imp |
|---|---|---|---|---|---|---|
| (Month) | (%) | (%)* | (%)* | (%) | (%) | (%) |
| 0 | 103 | 0.3 | 0.3 | ND | ND | 0.6 |
| 1 | 102 | 0.3 | 0.3 | ND | ND | 0.6 |
| 2 | 99 | 0.2 | 0.3 | ND | ND | 0.7 |
| 3 | 102 | 0.3 | 0.3 | ND | ND | 0.6 |
| 6 | 101 | 0.2 | 0.3 | 0.2 | ND | 0.8 |

*% Peak Area corrected for UV Response Factors: 1.06 for 5-Trans and 1.17 for 15-Keto

TABLE 5B
Accelerated Stability Data (40° C.) for Ocular Inserts Packages
with Oxygen Absorber (3-day hold)

| Time | Assay | 5-Trans | 15-Keto | RRT 0.61 | RRT 1.05 | Total Imp |
|---|---|---|---|---|---|---|
| (Month) | (%) | (%)* | (%)* | (%) | (%) | (%) |
| 1 | 102 | 0.3 | 0.3 | ND | ND | 0.6 |
| 2 | 101 | 0.2 | 0.3 | ND | ND | 0.7 |
| 3 | 104 | 0.3 | 0.4 | ND | ND | 0.8 |
| 6 | 104 | 0.2 | 0.4 | 0.2 | ND | 0.9 |

*% Peak Area corrected for UV Response Factors: 1.06 for 5-Trans and 1.17 for 15-Keto

Tensile force of ocular inserts with 4 mg bimatoprost was evaluated. The samples were stored at room temperature for 519 days and then accelerated aged at 55° C. for 3 days to reach an equivalent real time of 540 days (18 months). Tensile force of two different ocular inserts was tested in which the same 3-0 polypropylene monofilament suture was used. The tensile results from the samples were similar, as shown in Table 6.

TABLE 6
Tensile Force of Accelerated Aged (55° C.) Ocular Inserts

| Sample | Maximum Load (N) |
|---|---|
| 1 | 3.0 |
| 2 | 2.8 |
| 3 | 3.0 |
| 4 | 3.2 |
| 5 | 4.2 |
| 6 | 3.0 |
| 7 | 3.1 |
| 8 | 2.0 |
| 9 | 3.2 |
| 10 | 1.9 |
| Average | 2.9 ± 0.6 |

The shelf-life of the ocular inserts was estimated taking into consideration the stability and tensile force data above and the following assumptions:
a) The real-time stability data at 25° C./60% RH showed negligible product degradation relative to time zero within current analytical method variations.
b) The accelerated stability data at 40° C./75% RH showed 0.1% increase in 15-Keto level.
c) The tensile force data for accelerated aged Inserts at 55° C. indicated no deterioration at the suture weld.
d) Impurity level of 15-Keto reaching 1.0% limit (allowing for 4.0% impurities) was expected to be the rate-limiting factor.
e) 25° C. was considered the average real-time storage temperature for room temperature (15° C.-30° C.) for the purpose of shelf-life projection.
f) Real-time stability (25° C.) was projected per Arrhenius analysis assuming a 2-fold stability increase for every 10° C. lower temperature, e.g. a factor of 3 was used for projecting 25° C. stability from 40° C. data.
   The projected product shelf-life at 25° C. based on the 40° C./75% RH accelerated condition is as follows:
a) Per simple Arrhenius analysis: 6 months×3=18 months
b) Per extrapolation of %15-Keto reaching 1.0% limit at 25° C./60% RH:
   The %15-Keto did not appreciably grow between T=0 and 3 months for the accelerated sample within the reproducibility of the method (1.2% RSD).
   Even assuming that the 0.1% growth of the 15-Keto impurity from T=0 to 1 month for the R&D sample (real time) is accurate, it would take 36 months at this rate of growth to reach the specification limit of 4%.
   Furthermore, since the %15-Keto did not grow between time 0 and 6 months within analytical variations for the ocular inserts packages with oxygen absorber (real time), extrapolation for 0.7% growth from 0.3% (at time 0) to the ICH limit of 1.0% would result in an infinite shelf-life.

The projected shelf-life from time-zero is 18 months per Arrhenius analysis of 40° C./75% RH data and 36 months per available 25° C./60% RH data.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims. It will be understood by those skilled in the art that various changes in form and details may be made therein without departing from the scope of the invention encompassed by the appended claims.

1. A composition comprising a polymer matrix and stable amorphous bimatoprost, wherein the bimatoprost is dispersed in the polymer matrix.

2. The composition of claim 1, wherein the polymer matrix comprises a thermosetting or a thermoplastic polymer.

3.-7. (canceled)

8. The composition of claim 2, wherein the thermosetting polymer is silicone.

9. The composition of claim 8, wherein the silicone is MED-4810, MED-4820, MED-4830, MED-4840, MED-4842, MED1-4855, MED-4860, MED-4870, or MED-4880.

10. The composition of claim 1, wherein the composition is configured as a medical device.

11. (canceled)

12. The composition of claim 10, wherein the device has a ring shape.

13. The composition of claim 12, wherein the ring has a diameter of about 10 mm to about 40 mm and a cross-sectional thickness of about 0.1 mm to about 5 mm.

14. The composition of claim 13, wherein the diameter is about 20 mm to about 30 mm and the cross-sectional thickness is about 0.5 mm to about 1.5 mm.

15. The composition of claim 1, wherein the bimatoprost is about 1% to about 30% by weight of the composition.

16.-18. (canceled)

19. The composition of claim 15, wherein the bimatoprost is about 2%, about 3%, about 4%, about 5%, about 6%, about 7%, about 8%, about 10%, about 11%, about 12%, about 13%, about 14%, about 15%, about 16%, about 17%, about 18%, about 19%, about 20%, about 21%, or about 22% by weight of the composition.

20. The composition of claim 19, wherein the bimatoprost is about 2% or about 20% by weight of the composition.

21. (canceled)

22. A method of preparing a composition comprising:
dissolving bimatoprost in an organic solvent to form a solution;
mixing the solution with an uncured polymer;
removing the organic solvent; and
curing the polymer at a temperature above the melting point of bimatoprost.

23. The method of claim 22, wherein the curing temperature is equal to or above about 65° C., or equal to or above about 100° C.

24. (canceled)

25. The method of claim 23, wherein the curing temperature is about 140° C. to about 160° C.

26. The method of claim 22, wherein the polymer is silicone.

27. The method of claim 26, wherein the organic solvent is selected from dichloromethane, chloroform, and diethyl ether.

28. The method of claim 27, wherein the organic solvent is dichloromethane.

29. The method of claim 28, wherein the curing temperature is about 140° C. to about 160° C.

30. The method of claim 29, wherein the curing temperature is about 152° C.

31. The method of claim 30, wherein the curing step lasts for about 2 minutes to about 10 minutes.

32. The method of claim 31, wherein the curing step lasts for about 5 minutes.

33. The method of claim 32, wherein, before the curing step, the resulting mixture from the removing step is shaped.

34.-47. (canceled)

48. A ring shaped ocular insert comprising bimatoprost dispersed in a polymer matrix composition.

49. The ocular insert of claim 48, wherein the polymer matrix comprises a thermosetting polymer or a thermoplastic polymer.

50. The ocular insert of claim 49, wherein the thermosetting polymer is silicone.

51. The ocular insert of claim 50, wherein the silicone is MED-4810, MED-4820, MED-4830, MED-4840, MED-4842, MED1-4855, MED-4860, MED-4870, or MED-4880.

52. The ocular insert of claim 48, wherein the ring has a diameter of about 10 mm to about 40 mm and a cross-sectional thickness of about 0.1 mm to about 5 mm.

53. The ocular insert of claim 52, wherein the diameter is about 20 mm to about 30 mm and the cross-sectional thickness is about 0.5 mm to about 1.5 mm.

54. The ocular insert of claim 48, wherein the bimatoprost is about 1% to about 30% by weight of the composition.

55. The ocular insert of claim 54, wherein the bimatoprost is about 2%, about 3%, about 4%, about 5%, about 6%, about 7%, about 8%, about 10%, about 11%, about 12%, about 13%, about 14%, about 15%, about 16%, about 17%, about 18%, about 19%, about 20%, about 21%, or about 22% by weight of the composition.

56. The ocular insert of claim 55, wherein the bimatoprost is about 2% or about 20% by weight of the composition.

57. A method of lowering intraocular pressure comprising placement of a ring shaped ocular insert in eye of a subject in need thereof, wherein the ring shaped ocular insert comprises bimatoprost dispersed in a polymer matrix composition.

58. The method of claim 57, wherein the polymer matrix comprises a thermosetting polymer or a thermoplastic polymer.

59. The method of claim 58, wherein the thermosetting polymer is silicone.

60. The method of claim 59, wherein the silicone is MED-4810, MED-4820, MED-4830, MED-4840, MED-4842, MED1-4855, MED-4860, MED-4870, or MED-4880.

61. The method of claim 57, wherein the ring has a diameter of about 10 mm to about 40 mm and a cross-sectional thickness of about 0.1 mm to about 5 mm.

62. The method of claim 61, wherein the diameter is about 20 mm to about 30 mm and the cross-sectional thickness is about 0.5 mm to about 1.5 mm.

63. The method of claim 57, wherein the bimatoprost is about 1% to about 30% by weight of the composition.

64. The method of claim 63, wherein the bimatoprost is about 2%, about 3%, about 4%, about 5%, about 6%, about 7%, about 8%, about 10%, about 11%, about 12%, about 13%, about 14%, about 15%, about 16%, about 17%, about 18%, about 19%, about 20%, about 21%, or about 22% by weight of the composition.

65. The method of claim 64, wherein the bimatoprost is about 2% or about 20% by weight of the composition.
